SEQUENCE LISTING (1 sequence)

SEQ ID NO: 1 - protein, 585 aa, Unknown
  Bacteriophage
MGSSHHHHHHSSGLVPRGSHMSEAAQEAANAAEVAASQTQYYLKYFNPEIVYPKNARIMLDNGDIVRSTVVNNTSNPNVDMTGWVKVSSVSQIFDETYNITQSVINGNLITVDNFGAKGDGVTDDSAAFQAYCDSALTGQNLYLGAKGRYILKNQVDLKGKGLVGNGCGKVSEFYYNLGCIDVDGSSPDLQGKTAFINCGPTIQNLTARCSNGAGKQVSFIEIDGYLANIDHITLINFYNQIVVKQALVGFNFTNAWLYYSQNAGIYCEDPLNRVSTTGTFHNIYFQLGDGHAMIFDRDVHGCDFDNIIFESMNGGIKARTVAHCGFGKFWCENLKTATSKDWLEVTGANSCYGNSFTGYVKLLGGWTSKTSPTLDSLPTNNYGGVSVSAEGISIVNAGNKAKMLMLPSGFKTGNATIDETHISSSTVTPLVKRRVIGADSSGAQYLASDTYTKLSRKWGTYNHGSNNAGAFYAPMMLTYDQSFSTPQNNNGWKIVKESTGVYRVERVSGNTSVITNGHIVVGSPLMGSRLGTGTGATHGIQMIETYAGSWTSYTEAAGFKVFWRDSSNALVDPHRFTVAFTATS

(19) United States
(12) Patent Application Publication
(10) Pub. No.: US 2021/0121519 A1
(43) Pub. Date: Apr. 29, 2021

(54) USE OF TAIL FIBER PROTEIN IN THE PREVENTION OF ACINETOBACTER BAUMANNII INFECTIONS

(71) Applicant: TZU CHI UNIVERSITY, HUALIEN (TW)

(72) Inventor: NIEN-TSUNG LIN, HUALIEN (TW)

(21) Appl. No.: 16/733,590

(22) Filed: Jan. 3, 2020

(30) Foreign Application Priority Data
Oct. 29, 2019 (TW) 108139082

(51) Int. Cl. A61K 38/16; A61P 31/04

(52) U.S. Cl. CPC A61K 38/162; A61P 31/04

(57) ABSTRACT

A use of a tail fiber protein in the prevention of Acinetobacter baumannii infections is disclosed. The tail fiber protein from bacteriophages is coated or sprayed on carriers (such as pipelines and medical devices in hospitals) to inhibit biofilm formation of Acinetobacter baumannii and further prevent Acinetobacter baumannii infections.

### SEQUENCE LISTING

This application includes as part of its disclosure a biological sequence listing text file named “3315-1142-Sequence-Listing.txt” having a size 4,779 bytes that was created Jun. 3, 2020, which is hereby incorporated by reference in its entirety.

### BACKGROUND OF THE INVENTION

### Field of the Invention

The present invention relates to a use of a tail fiber protein, especially to a use of a tail fiber protein in the prevention of Acinetobacter baumannii infections.

### Description of Related Art

Acinetobacter baumannii, abbreviated as AB, is a rod-shaped Gram-negative bacterium with low motility due to lack of flagella. They are with strong vitality, naturally occurring in environmental matrices such as soil, water, food, etc. broadly and permanently, or even human skin or mucosa.

Acinetobacter baumannii can live in human bodies without causing infections or symptoms, and mainly colonize on skin, underarm, conjunctiva, perineum, oral cavity, upper respiratory tract and lower gastrointestinal tract, sometimes also in throat saliva, and mucosa. This bacteria presents in the normal flora of about 10% health people.

Moreover, this highly-hydrophilic organism thrives in warm and wet environments. In addition to medical pipelines in the intensive care unit (ICU) including pipes of medical ventilators, Foley catheters, chest tubes, central venous catheters, surgical drainages, etc., Acinetobacter baumannii are commonly found on trolleys, sinks, beds and mattress, even air in the hospitals.

Most of scientists think the organism is not pathogenic when Acinetobacter baumannii was isolated for the first time. Along with the fast development of the medical technology, various invasive catheters have been developed and used in critically ill patients. These catheters provide optimal environmental conditions to Acinetobacter baumannii and the critically ill patients have weakened immune systems. Thus the frequency of A. baumannii has been increased gradually.

Cultures of sputum, urine, blood, and wound exudate are done for diagnosis of A. baumannii in critically ill patients. Once the bacteria are detected in the patient's sputum, the positive result may be resulted from the contaminated specimen and the treatment is not necessary. Yet if A. baumannii is observed in the urine, blood, and wound exudate cultures, the result shows that the patients are infected. Especially the positive result in the blood culture, this represents the bacteria already cause severe infections in patients, and even trigger lethal sepsis.

Risk factors for A. baumannii infections include immune function of the patient, disease severity, etc. Thus the more serious the illness, the longer hospital stays, with medical ventilators, and the more catheters used, the greater frequency of infections in patients. Thereby the critically ill patients in the intensive care unit (ICU) are at high risk for nosocomial infection.

In healthy people, A. baumannii is a common flora. A. baumannii can be spread by person-to-person contact or contact with contaminated surfaces of general medical devices. Although not as horrifying as droplet transmission, A. baumannii is still easily transmitted once medical professionals forget to clean their hands before in contact with the next patient.

A. baumannii, infection may cause bacteremia, sepsis, pneumonia, meningitis, peritonitis, endocarditis, urinary tract infection, skin infection, etc., sometimes even cause death of patients. The mortality rate of patients with A. baumannii, have been reported to be as highly as 20-50%.

Furthermore, higher antibiotic resistance rates for A. baumannii are resulted from the abuse of antibiotics. The use of antibiotics should be reframed. In recent years, more and more antibiotic resistant strains of antibiotic resistant strains are appearing and this poses a greater challenge in treatment of patients with these antibiotic resistant strains.

For A. baumannii nosocomial infection, complete infection control and prevention are far more important than the treatment. Thus there is a room for improvement and there is a need to provide a novel way for prevention of A. baumannii infections caused by A. baumannii in general medical devices.

### SUMMARY OF THE INVENTION

Therefore it is a primary object of the present invention to provide a use of a tail fiber protein in the prevention of Acinetobacter baumannii infections. The tail fiber protein derived from bacteriophage ϕAB6 is disposed on carriers such as pipelines and medical devices in the hospitals to degrade, inhibit and prevent biofilm formation of Acinetobacter baumannii and further prevent Acinetobacter baumannii infections.

In order to achieve the above object, a use of a tail fiber protein in the prevention of Acinetobacter baumannii infections according to the present invention is provided. The amino acid sequence of the tail fiber protein is set forth in SED ID NO: 1 and the tail fiber protein is disposed on carriers to prevent Acinetobacter baumannii infections associated with the carriers.

Preferably, regarding the use of the tail fiber protein in the prevention of Acinetobacter baumannii infections, the tail fiber protein is obtained from a bacteriophage.

Preferably, regarding the use of the tail fiber protein in the prevention of Acinetobacter baumannii infections, the Acinetobacter baumannii is A. baumannii strain 54149.

Preferably, regarding the use of the tail fiber protein in the prevention of Acinetobacter baumannii infections, the tail fiber protein is able to degrade a biofilm of Acinetobacter baumannii.

Preferably, regarding the use of the tail fiber protein in the prevention of Acinetobacter baumannii infections, the tail fiber protein is able to inhibit biofilm formation of Acinetobacter baumannii.

Preferably, regarding the use of the tail fiber protein in the prevention of Acinetobacter baumannii infections, the tail fiber protein is able to reduce thickness of a biofilm of Acinetobacter baumannii.

Preferably, regarding the use of the tail fiber protein in the prevention of Acinetobacter baumannii infections, the tail fiber protein is disposed on the carrier by coating.

Preferably, regarding the use of the tail fiber protein in the prevention of Acinetobacter baumannii infections, the tail fiber protein is arranged at the carrier by spraying.

Preferably, regarding the use of the tail fiber protein in the prevention of Acinetobacter baumannii infections, the carrier is selected from one of the followings: a pipe of the ventilator, a Foley catheter, a chest tube, a central venous catheter, and a surgical drainage.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein:

FIG. 1 is a bar chart showing biofilm degradation results of an embodiment according to the present invention;

FIG. 2 is a bar chart showing biofilm inhibition effect of an embodiment according to the present invention;

FIG. 3 shows images showing biofilm thickness of an embodiment according to the present invention;

FIG. 4A-4B show experiment results of an embodiment according to the present invention.

### DETAILED DESCRIPTION OF THE PREFERRED EMBODIMENT

In order to learn features and functions of the present invention, please refer to the following embodiments and related figures.

Acinetobacter baumannii infections typically occur in people in hospitals and even threaten patient's lives. In view of this, the present invention provides a use of a tail fiber protein in the prevention of Acinetobacter baumannii infections to solve the problems caused by the infections.

The features, related structure and methods used are described in tails in the following embodiments.

The tail fiber protein (abbreviated as TFP) used in the present invention is derived from bacteriophage ϕAB6 and having amino acid sequence set forth in SED ID NO: 1 as below:

MGSSHHHHHHSSGLVPRGSHMSEAAQEAANAAEVAASQTQYYL
KYFNPEIVYPKNARIMLDNGDIVRSTVVNNTSNPNVDMTGWVKV
SSVSQIFDETYNITQSVINGNLITVDNFGAKGDGVTDDSAAFQAYC
DSALTGQNLYLGAKGRYILKNQVDLKGKGLVGNGCGKVSEFYY
NLGCIDVDGSSPDLQGKTAFINCGPTIQNLTARCSNGAGKQVSFIEI
DGYLANIDHITLINFYNQIVVKQALVGFNFTNAWLYYSQNAGIYC
EDPLNRVSTTGTFHNIYFQLGDGHAMIFDRDVHGCDFDNIIFESMN
GGIKARTVAHCGFGKFWCENLKTATSKDWLEVTGANSCYGNSFT
GYVKLLGGWTSKTSPTLDSLPTNNYGGVSVSAEGISIVNAGNKAK
MLMLPSGFKTGNATIDETHISSSTVTPLVKRRVIGADSSGAQYLAS
DTYTKLSRKWGTYNHGSNNAGAFYAPMMLTYDQSFSTPQNNNG
WKIVKESTGVYRVERVSGNTSVITNGHIVVGSPLMGSRLGTGTGA
THGIQMIETYAGSWTSYTEAAGFKVFWRDSSNALVDPHRFTVAFT
ATS.

The tail fiber protein is further disposed on a carrier to prevent the carrier from being contaminated with Acinetobacter baumannii. In this embodiment, the Acinetobacter baumannii is mild antibiotic-resistant A. baumannii strain 54149. The tail fiber protein is obtained by using Polymerase chain reaction (PCR). The purified ORF40ϕAB6 segment in bacteriophage (ϕAB6) with polysaccharide depolymerase activity is amplified and extracted while the primers used include XhoI_ABTF6_R (5′-CTCGAGTTAACTCGTTGCTGTAAATGC-3′) and NdeI_ABTF6_F (5′-CATATGAGTGAAGCTGCTGCTCAAGAGGCTGC-3′). Next the segment cut by restriction enzymes XhoI and NdeI is inserted into the pET-28a vector and the pET-28a vector is delivered into Escherichia coli BL21 (DE3). Lastly the protein obtained is analyzed and purified.

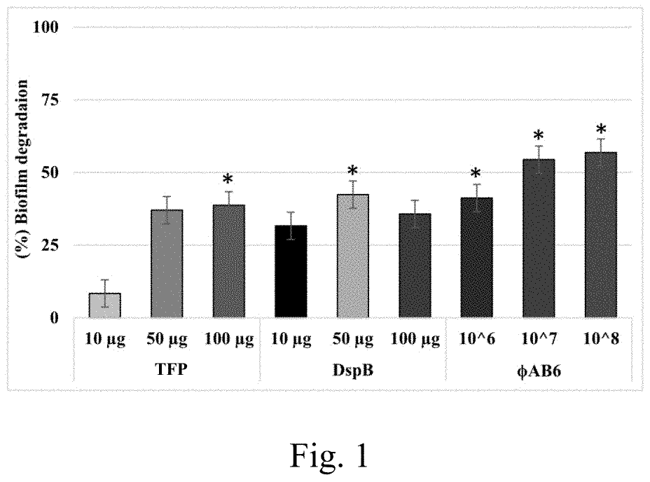

Refer to FIG. 1, a bar chart showing biofilm degradation results of an embodiment is revealed. A sterile polystyrene microtiter plate (microplate) with 96 wells is used to perform biofilm degradation tests. A. baumannii strain 54149 is cultured in the microplate with culture medium for 48 hours. After bacterial suspension (A. baumannii strain 54149 and culture medium) being removed, add bacteriophage (ϕAB6) at different concentrations (10⁶, 10⁷, and 10⁸ PFU), the tail fiber protein (TFP) at different concentrations (10, 50, and 100 μg), and DspB at different concentrations (10, 50, and 100 μg) respectively into the microplate and incubate at 37° C., 150 rpm for 4 hours. DspB, also known as Dispersin B, is an anti-biofilm agent that could be used in combination with antibiotics for the treatment of bacterial infections. Next wash with 1× phosphate buffered saline (PBS) gently and stain with 200 μl, 1, 0.1% crystal violet solution for 30 minutes. Then remove the crystal violet solution with 1×PBS and let the microplate dry at 55° C. for 30 minutes. Lastly add 95% alcohol for dissolution, shake the microplate for 10 minutes, and determine biofilm degradation by measuring absorbance of both attached cells and unattached cells (using a ELISA reader at OD 570 nm). The average absorbance of the respective groups (the bacteriophage, the tail fiber protein, and DspB) being processed is divided by the average absorbance of the control wells and then is multiplied by 100 to learn the biofilm degradation effect (biofilm degradation rate).

As shown in FIG. 1, the biofilm degradation rate of the tail fiber protein (TFP) at the concentration of 100 μg is up to 36% (p<0.05).

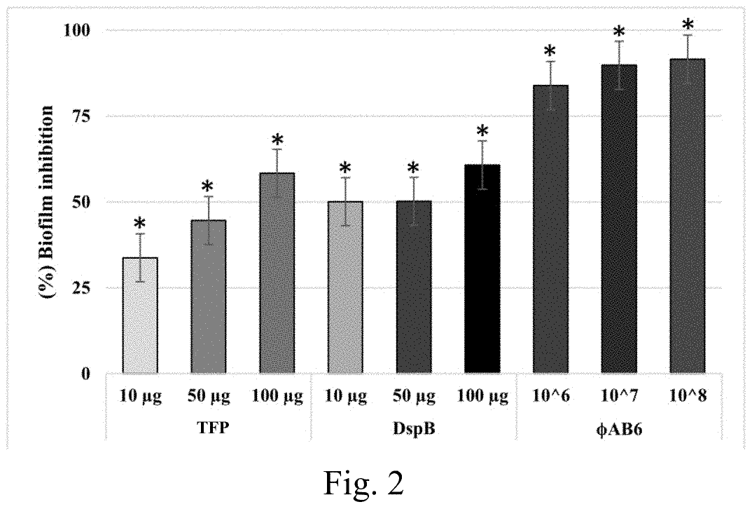

Refer to FIG. 2, a bar chart showing biofilm inhibition effect of an embodiment is disclosed. Biofilm inhibition tests are carried out by using the same sterile polystyrene microtiter plate (microplate) with 96 wells mentioned above. Bacteriophage (ϕAB6) at different concentrations (10⁶, 10⁷, and 10⁸ PFU), the tail fiber protein (TFP) at different concentrations (10, 50, and 100 μg), DspB at different concentrations (10, 50, and 100 μg) and A. baumannii strain 54149 (abbreviated as Ab-54149) are respectively added into and incubated in the microplate and incubate for 24 hours. Then the biofilm inhibition effect is evaluated by measuring absorbance of unattached cells (at OD 570 nm in an ELISA reader). The biofilm inhibition rate is calculated by the average absorbance of respective groups (the bacteriophage, the tail fiber protein, and DspB) processed being divided by the average absorbance of the control wells and then being multiplied by 100.

As shown in FIG. 2, the tail fiber protein (TFP) significantly inhibits the biofilm formation of the A. baumannii strain 54149 (p<0.01) and the biofilm inhibition rate at the concentration of 100 μg is up to 60% (p <0.05). Moreover, the biofilm inhibition effect is proportional to the concentration of the tail fiber protein (TFP). The results show that the tail fiber protein (TFP) has a great ability in inhibition of biofilm formation.

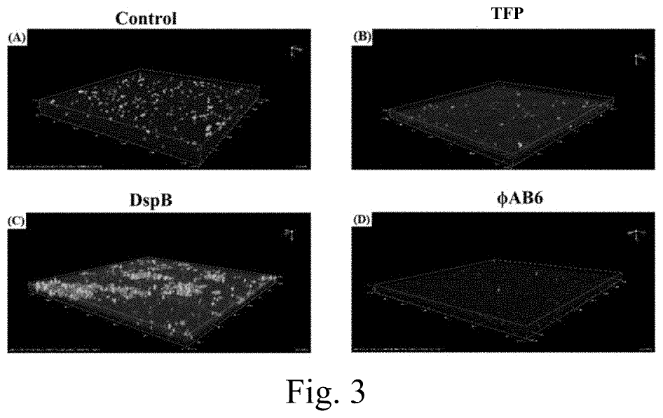

Refer to FIG. 3, the images showing biofilm thickness are disclosed. The confocal Laser Scanning Microscope (CLSM) is used to observe 3-dimensional structure of the biofilm for testing a reduction of biofilm thickness. The biofilm of the A. baumannii strain 54149 is cultured on round plastic coverslips in a 24-well microplate. Then wash the round plastic coverslips twice with 1×PBS and add the bacteriophage (ϕAB6, 10⁸ PFU in PBS), the tail fiber protein (TFP, 100 μg in PBS) and DspB (100 μg in PBS) into the plastic coverslips to be incubated at 37° C., 150 rpm for 4 hours. After being washed with 1×PBS, stain in a dark room with wheat germ agglutinin-Alexa Fluor (WGA-AF) for 2 hours. The Alexa Fluor can bind to extracellular polymeric substances (EPS) to emit green fluorescence. At last, CLSM is used to observe the biofilm after washing with PBS.

As shown in FIG. 3, biofilm thickness reduction is shown. Compared to the control group (Group A, the tail fiber protein is inactivated), the thickness of the biofilm in the group B being processed by the tail fiber protein is significantly reduced. Moreover, compared to the present invention (group B), the thickness of the biofilm of A. baumannii strain 54149 on the cover slip of the group treated by DspB (Group C) is not reduced effectively.

Furthermore, the present tail fiber protein is disposed on the carrier by coating and the carrier is selected from, but not limited to, one of the followings: a pipe of the ventilator, a Foley catheter, a chest tube, a central venous catheter, and a surgical drainage. The present tail fiber protein can also be arranged at the carrier by spraying. Besides the pipelines mentioned above, the carrier also includes medical devices used in hospitals such as wheelchairs, beds, trolleys, door handles, etc. The tail fiber protein is disposed over the devices by spraying to prevent biofilm formation of A. baumannii strain 54149 and related infections.

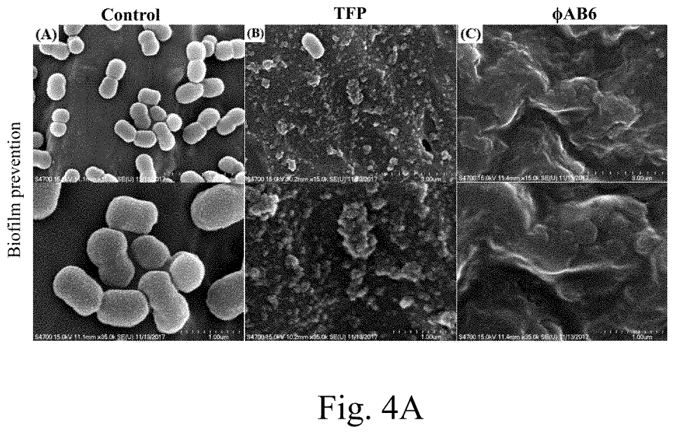

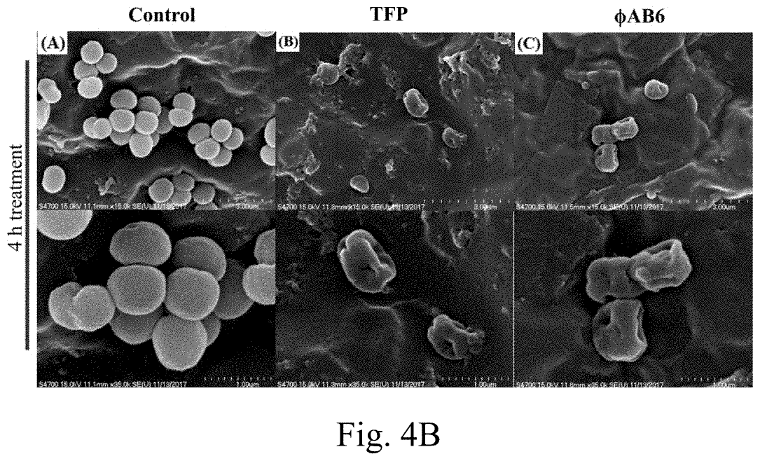

Refer to FIG. 4A and FIG. 4B, experiment results of an embodiment of the present invention are revealed. A scanning electron microscope (SEM) is used to observe the biofilm on a catheter (Foley catheter with the silicon coating). Three Foley catheters with the silicon coating are respectively immersed in 1 ml 1×PBS (Group A as the control group), 1 mg tail fiber protein (Group B, TFP) and 10⁸ PFU bacteriophage (Group C, ϕAB6) for 16 hours. Then the Foley catheters with the silicon coating are immersed in bacteria suspension (A. baumannii strain 54149+culture medium). Next wash twice with 1×PBS and use 2.5% (w/v) Glutaraldehyde in 01.M cacodylate buffer and 1% aqueous osmium tetroxide for fixation. Then wash twice with 1×PBS again and dehydrate in a graded ethanol series (50%, 70% and 95%). Lastly perform chemical drying by using 100% Hexamethyldisilazane (HMDS). Then the scanning electron microscope (SEM) is used to observe the results of the experiment designed to test the prevention effect of the tail fiber protein on biofilm formation of A. baumannii strain 54149 on the Foley catheter with silicon coating.

As shown in FIG. 4A, A. baumannii strain 54149 is unable to grow on the catheters already being immersed in the tail fiber protein (TFP) and the bacteriophage (ϕAB6). Thereby the results indicate that the tail fiber protein (TFP) can prevent both the growth of A. baumannii strain 54149 and its biofilm formation.

Refer to FIG. 4B, the experiment results that show biofilm degradation effect of the tail fiber protein (TFP) on A. baumannii strain 54149 on the catheter are disclosed. After being immersed in bacteria suspension (A. baumannii strain 54149+culture medium) at 37° C. for 48 hours, the Foley catheters with the silicon coating are respectively further treated by 1×PBS(Group A as the control group), 1 mg tail fiber protein (Group B, TFP) and 10⁸ PFU bacteriophage (Group C, ϕAB6) for 4 hours. The same as the experiment in FIG. 4A, the scanning electron microscope (SEM) is used to observe the biofilm. As shown in FIG. 4B, the tail fiber protein (TFP) degrades the biofilm of A. baumannii strain 54149 to prevent A. baumannii strain 54149 from growing.

According to the above experiments, it is proved that the tail fiber protein (TFP) can not only prevent biofilm formation of A. baumannii strain 54149 on catheters or other medical devices and related infections, the tail fiber protein (TFP) can also degrade the biofilm already formed (by A. baumannii strain 54149) and stop the growth of A. baumannii strain 54149.

The tail fiber protein of the present invention prevents the infections of A. baumannii strain 54149 by inhibiting biofilm formation of A. baumannii strain 54149 on the carrier. In the conventional way, bacteriophages which directly lyse and burst A. baumannii strain 54149 are used so that bacterial endotoxin is released into human bodies through the carrier and causing a wide range of adverse effects on the bodies. The way the present invention uses is quite different from the conventional way.

In summary, besides reducing attachment and colonization of A. baumannii strain 54149 on the carrier, the present tail fiber protein also reduces antibiotic tolerance of the bacteria strain and solves the problem of endotoxin release while A. baumannii strain 54149 being killed.

Additional advantages and modifications will readily occur to those skilled in the art. Therefore, the invention in its broader aspects is not limited to the specific details, and representative devices shown and described herein. Accordingly, various modifications may be made without departing from the spirit or scope of the general inventive concept as defined by the appended claims and their equivalent.

1-3. (canceled)

4. A use of a tail fiber protein in the prevention of Acinetobacter baumannii infections; wherein amino acid sequence of the tail fiber protein is set forth in SEQ ID NO: 1 and the tail fiber protein is disposed on a carrier to prevent Acinetobacter baumannii infections associated with the carrier; wherein the tail fiber protein is able to degrade a biofilm of Acinetobacter baumannii.

5. A use of a tail fiber protein in the prevention of Acinetobacter baumannii infections; wherein amino acid sequence of the tail fiber protein is set forth in SEQ ID NO: 1 and the tail fiber protein is disposed on a carrier to prevent Acinetobacter baumannii infections associated with the carrier; wherein the tail fiber protein is able to inhibit biofilm formation of Acinetobacter baumannii.

6. A use of a tail fiber protein in the prevention of Acinetobacter baumannii infections; wherein amino acid sequence of the tail fiber protein is set forth in SEQ ID NO: 1 and the tail fiber protein is disposed on a carrier to prevent Acinetobacter baumannii infections associated with the carrier; wherein the tail fiber protein is able to reduce thickness of a biofilm of Acinetobacter baumannii.

7-9. (canceled)
